(19) United States
(12) Patent Application Publication
(10) Pub. No.: US 2024/0099380 A1
(43) Pub. Date: Mar. 28, 2024

(54) AEROSOL PROVISION DEVICE WITH INDUCTION COILS

(71) Applicant: NICOVENTURES TRADING LIMITED, London (GB)

(72) Inventor: Sioned OWEN, Brighton Sussex (GB)

(21) Appl. No.: 18/262,979

(22) PCT Filed: Feb. 2, 2022
(86) PCT No.: PCT/EP2022/052397

§ 371 Date: Jul. 26, 2023

(30) Foreign Application Priority Data
Feb. 3, 2021 (GB) 2101485.7

(51) Int. Cl. A24F 40/465; A24F 40/20; H05B 6/10

(52) U.S. Cl. CPC A24F 40/465; A24F 40/20; H05B 6/108

(57) ABSTRACT

A non-combustible aerosol provision device for generating aerosol from aerosol-generating material can include an aerosol-generation zone for receiving one or more consumables including aerosol-generating material, an aerosol generator configured to cause the aerosol to be generated from the one or more consumables including aerosol-generating material in the aerosol-generation zone, and an engagement region for engagement and disengagement with at least one user-insertable support for supporting the one or more consumables comprising aerosol-generating material in the aerosol-generation zone.

### PRIORITY CLAIM

The present application is a National Phase entry of PCT Application No. PCT/EP2022/052397, filed Feb. 2, 2022, which claims priority from GB Application No. 2101485.7, filed Feb. 3, 2021, each of which is hereby fully incorporated herein by reference.

### TECHNICAL FIELD

The present disclosure relates to apparatus (non-combustible aerosol provision devices) for generating aerosol from aerosol-generating material, methods of configuring apparatus (non-combustible aerosol provision devices) for generating aerosol from aerosol-generating material, and non-combustible aerosol provision systems for generating aerosol from aerosol-generating material.

### BACKGROUND

Smoking articles such as cigarettes, cigars and the like burn tobacco during use to create tobacco smoke. Attempts have been made to provide alternatives to these articles by creating products that release compounds without combusting. Examples of such products are so-called “heat not burn” products or tobacco heating devices or products, which release compounds by heating, but not burning, material. The material may be, for example, tobacco or other non-tobacco products, which may or may not contain nicotine.

### SUMMARY

A first aspect of the present disclosure provides a non-combustible aerosol provision device for generating aerosol from aerosol-generating material, the non-combustible aerosol provision device comprising: an aerosol generating aerosol-generation zone for receiving one or more consumables comprising aerosol-generating material; an aerosol generator configured to cause the aerosol to be generated from the one or more consumables comprising aerosol-generating material in the aerosol-generation zone; and an engagement region for engagement and disengagement with at least one user-insertable support for supporting the one or more consumables comprising aerosol-generating material in the aerosol-generation zone.

In an exemplary embodiment, the aerosol generator is or comprises a heating device for use in heating the one or more consumables comprising aerosol-generating material in the aerosol-generation zone.

In some embodiments, the heating device comprises a magnetic field generator for generating one or more varying magnetic fields that penetrate one or more respective longitudinal portions of the aerosol-generation zone in use.

In an exemplary embodiment, the magnetic field generator comprises a plurality of flat spiral coils of electrically-conductive material arranged sequentially and in respective planes along a longitudinal axis of the aerosol-generation zone, such as a heating zone.

In an exemplary embodiment, the aerosol-generation zone extends through a hole in each of the plurality of flat spiral coils. In some embodiments, the hole in each of the plurality of flat spiral coils is at the center of the respective flat spiral coil.

In an exemplary embodiment, the non-combustible aerosol provision device comprises a heating element comprising heating material that is heatable by penetration with a varying magnetic field generatable by the magnetic field generator to heat the aerosol-generation zone.

In an exemplary embodiment, the heating element is tubular and configured to be around the one or more consumables comprising aerosol-generating material when the one or more consumables are received by the aerosol-generation zone.

In an exemplary embodiment, the heating element is a male member and configured to penetrate the one or more consumables comprising aerosol-generating material when the one or more consumables are received by the aerosol-generation zone.

In an exemplary embodiment, the heating element is encircled by the aerosol-generation zone.

In an exemplary embodiment, the engagement region is configured for slidable engagement and disengagement with the at least one user-insertable support.

In an exemplary embodiment, the aerosol-generating material comprises tobacco and/or is reconstituted and/or is in the form of a gel and/or comprises an amorphous solid.

In an exemplary embodiment, the heating material comprises one or more materials selected from the group consisting of: an electrically-conductive material, a magnetic material, and a magnetic electrically-conductive material.

In an exemplary embodiment, the heating material comprises a metal or a metal alloy.

In an exemplary embodiment, the heating material comprises one or more materials selected from the group consisting of: aluminum, gold, iron, nickel, cobalt, conductive carbon, graphite, steel, plain-carbon steel, mild steel, stainless steel, terrific stainless steel, molybdenum, silicon carbide, copper, and bronze.

In an exemplary embodiment, the aerosol-generating material comprises tobacco and/or is reconstituted and/or is in the form of a gel and/or comprises an amorphous solid.

A second aspect of the present disclosure provides a non-combustible aerosol provision system for generating aerosol from aerosol-generating material, the non-combustible aerosol provision system comprising: at least one user-insertable support; a non-combustible aerosol provision device comprising: an aerosol-generation zone for receiving one or more consumables comprising aerosol-generating material; an aerosol generator, such as a heating device, configured to cause the aerosol to be generated from the one or more consumables comprising aerosol-generating material in the aerosol-generation zone; and an engagement region; wherein the at least one user-insertable support is engageable and disengageable with the engagement region and is for supporting the one or more consumables comprising aerosol-generating material in the aerosol-generation zone.

In an exemplary embodiment, the at least one user-insertable support comprises a passageway to form at least a part of the aerosol-generation zone for receiving the one or more consumables comprising aerosol-generating material.

In an exemplary embodiment, the aerosol generator is or comprises a heating device for use in heating the one or more consumables comprising aerosol-generating material in the aerosol-generation zone.

In some embodiments, the heating device comprises a magnetic field generator for generating one or more varying magnetic fields that penetrate one or more respective longitudinal portions of the aerosol-generation zone in use.

In an exemplary embodiment, the magnetic field generator comprises a plurality of flat spiral coils of electrically-conductive material arranged sequentially and in respective planes along a longitudinal axis of the aerosol-generation zone.

In an exemplary embodiment, the aerosol-generation zone extends through a hole in each of the plurality of flat spiral coils. In some embodiments, the hole in each of the plurality of flat spiral coils is at a center of the respective flat spiral coil.

In an exemplary embodiment, the non-combustible aerosol provision system comprises a heating element comprising heating material that is heatable by penetration with a varying magnetic field generatable by a magnetic field generator to heat the aerosol-generation zone.

In an exemplary embodiment, the non-combustible aerosol provision device comprises the heating element.

In an exemplary embodiment, the heating element is a male member and configured to penetrate the one or more consumables comprising aerosol-generating material when the one or more consumables are received by the aerosol-generation zone.

In an exemplary embodiment, the at least one user-insertable support comprises an opening communicable with a passageway through which the heating element as a male member is insertable along an insertion path. In an exemplary embodiment, the insertion path comprises a portion that is non-parallel to a longitudinal axis of the aerosol-generation zone. In an exemplary embodiment, the insertion path is entirely non-parallel to a longitudinal axis of the aerosol-generation zone.

In an exemplary embodiment, the non-combustible aerosol provision system comprises the one or more consumables comprising aerosol-generating material and wherein the one or more consumables comprises the heating element.

In an exemplary embodiment, the non-combustible aerosol provision system comprises a first user-insertable support and a second user-insertable support, wherein the first user-insertable support and second user-insertable support are interchangeably insertable in the non-combustible aerosol provision device by a user of the non-combustible aerosol provision system. In an exemplary embodiment, the first user-insertable support comprises a first passageway to form at least a part of a first aerosol-generation zone and the second user-insertable support comprises a second passageway to form at least a part of a second aerosol-generation zone and wherein the first passageway is geometrically different to the second passageway.

In an exemplary embodiment, the first user-insertable support and a first heating element form a first set of components and the second user-insertable support and a second heating element form a second set of components, wherein the first heating element and the second heating element each comprise heating material that is heatable by penetration with a varying magnetic field generatable by a magnetic field generator to heat the respective first and second aerosol-generation zones.

In an exemplary embodiment, the first heating element is tubular and comprises a first tubular passageway and the second heating element is tubular and comprises a second tubular passageway and wherein the first tubular passageway and second tubular passageways are geometrically different.

In an exemplary embodiment, the heating material comprises one or more materials selected from the group consisting of: an electrically-conductive material, a magnetic material, and a magnetic electrically-conductive material.

In an exemplary embodiment, the heating material comprises a metal or a metal alloy.

In an exemplary embodiment, the heating material comprises one or more materials selected from the group consisting of: aluminum, gold, iron, nickel, cobalt, conductive carbon, graphite, steel, plain-carbon steel, mild steel, stainless steel, terrific stainless steel, molybdenum, silicon carbide, copper, and bronze.

In an exemplary embodiment, the aerosol-generating material comprises tobacco and/or is reconstituted and/or is in the form of a gel and/or comprises an amorphous solid.

A third aspect of the present disclosure provides a method of configuring a non-combustible aerosol provision device for generating aerosol from aerosol-generating material, the method comprising: providing a non-combustible aerosol provision device for generating the aerosol from aerosol-generating material; and user engagement of an engagement region of the non-combustible aerosol provision device with at least one user-insertable support for supporting one or more consumables comprising aerosol-generating material in an aerosol-generation zone of the non-combustible aerosol provision device.

In an exemplary embodiment, the aerosol-generating material comprises tobacco and/or is reconstituted and/or is in the form of a gel and/or comprises an amorphous solid.

In an exemplary embodiment, the method comprises heating a heating element of the non-combustible aerosol provision device.

In an exemplary embodiment, the heating element comprises heating material and the heating comprises heating the heating material by penetration with a varying magnetic field generated by a magnetic field generator to heat the aerosol-generation zone.

In an exemplary embodiment, the heating material comprises one or more materials selected from the group consisting of: an electrically-conductive material, a magnetic material, and a magnetic electrically-conductive material.

In an exemplary embodiment, the heating material comprises a metal or a metal alloy.

In an exemplary embodiment, the heating material comprises one or more materials selected from the group consisting of: aluminum, gold, iron, nickel, cobalt, conductive carbon, graphite, steel, plain-carbon steel, mild steel, stainless steel, terrific stainless steel, molybdenum, silicon carbide, copper, and bronze.

A fourth aspect of the present disclosure provides a non-combustible aerosol provision system for generating aerosol from aerosol-generating material, the non-combustible aerosol provision system comprising: an aerosol-generator, such as a heater, for use in generating aerosol from one or more consumables comprising aerosol-generating material; and a plurality of adapters interchangeably couplable to the aerosol generator by a user of the non-combustible aerosol provision system; wherein each adapter of the plurality of adapters is differently dimensioned to support a respectively differently dimensioned consumable comprising aerosol-generating material in the aerosol generator.

In an exemplary embodiment, the aerosol generator comprises a plurality of spiral coils of electrically-conductive material spaced apart from each other along an axis of the aerosol generator for generating respective varying magnetic fields.

In an exemplary embodiment, the aerosol generator is a heater for heating the aerosol-generating material in thermal contact with the heater. In an exemplary embodiment the plurality of spiral coils of electrically-conductive material are configured to cause heating of the aerosol-generating material in use.

### BRIEF DESCRIPTION OF THE DRAWINGS

Embodiments of the disclosure will now be described, by way of example only, with reference to the accompanying drawings, in which:

FIG. 1 shows a schematic perspective view of an example of a non-combustible aerosol provision device for generating aerosol from aerosol-generating material.

FIG. 2 shows a schematic side view of the example device of FIG. 1 when combinable with a consumable comprising aerosol-generating material to form a non-combustible aerosol provision system.

FIG. 3 shows an enlarged schematic side view of part of FIG. 2.

FIG. 4 shows an example of a user-insertable support with an opening communicable with a passageway.

FIG. 5 shows a flow diagram showing an example of a method of configuring a non-combustible aerosol provision device for generating aerosol from aerosol-generating material.

### DETAILED DESCRIPTION OF THE DRAWINGS

Aerosol-generating material is a material that is capable of generating aerosol, for example when heated, irradiated or energized in any other way. Aerosol-generating material may, for example, be in the form of a solid, liquid or gel which may or may not contain an active substance and/or flavorants. In some embodiments, the aerosol-generating material may comprise an “amorphous solid”, which may alternatively be referred to as a “monolithic solid” (i.e. non-fibrous). In some embodiments, the amorphous solid may be a dried gel. The amorphous solid is a solid material that may retain some fluid, such as liquid, within it. In some embodiments, the aerosol-generating material may for example comprise from about 50 wt %, 60 wt % or 70 wt % of amorphous solid, to about 90 wt %, 95 wt % or 100 wt % of amorphous solid.

The aerosol-generating material may comprise one or more active substances and/or flavors, one or more aerosol-former materials, and optionally one or more other functional material.

The aerosol-former material may comprise one or more constituents capable of forming an aerosol. In some embodiments, the aerosol-former material may comprise one or more of glycerine, glycerol, propylene glycol, diethylene glycol, triethylene glycol, tetraethylene glycol, 1,3-butylene glycol, erythritol, meso-Erythritol, ethyl vanillate, ethyl laurate, a diethyl suberate, triethyl citrate, triacetin, a diacetin mixture, benzyl benzoate, benzyl phenyl acetate, tributyrin, lauryl acetate, lauric acid, myristic acid, and propylene carbonate.

The one or more other functional materials may comprise one or more of pH regulators, coloring agents, preservatives, binders, fillers, stabilizers, and/or antioxidants.

The material may be present on or in a support, to form a substrate. The support may, for example, be or comprise paper, card, paperboard, cardboard, reconstituted material, a plastics material, a ceramic material, a composite material, glass, a metal, or a metal alloy. In some embodiments, the support comprises a susceptor. In some embodiments, the susceptor is embedded within the material. In some alternative embodiments, the susceptor is on one or either side of the material.

An aerosol-modifying agent is a substance, typically located downstream of the aerosol generation area, that is configured to modify the aerosol generated, for example by changing the taste, flavor, acidity or another characteristic of the aerosol. The aerosol-modifying agent may be provided in an aerosol-modifying agent release component, that is operable to selectively release the aerosol-modifying agent.

The aerosol-modifying agent may, for example, be an additive or a sorbent. The aerosol-modifying agent may, for example, comprise one or more of a flavorant, a colorant, water, and a carbon adsorbent. The aerosol-modifying agent may, for example, be a solid, a liquid, or a gel. The aerosol-modifying agent may be in powder, thread or granule form. The aerosol-modifying agent may be free from filtration material.

A consumable is an article comprising or consisting of aerosol-generating material, part or all of which is intended to be consumed during use by a user. A consumable may comprise one or more other components, such as an aerosol-generating material storage area, an aerosol-generating material transfer component, an aerosol generation area, a housing, a wrapper, a mouthpiece, a filter and/or an aerosol-modifying agent. A consumable may also comprise an aerosol generator, such as a heater, that emits heat to cause the aerosol-generating material to generate aerosol in use. The heater may, for example, comprise combustible material, a material heatable by electrical conduction, or a susceptor.

As used herein, the term “delivery system” is intended to encompass systems that deliver at least one substance to a user, and includes: non-combustible aerosol provision systems that release compounds from an aerosol-generating material without combusting the aerosol-generating material, such as electronic cigarettes, tobacco heating products, and hybrid systems to generate aerosol using a combination of aerosol-generating materials.

According to the present disclosure, a “non-combustible” aerosol provision system is one where a constituent aerosol-generating material of the aerosol provision system (or component thereof) is not combusted or burned in order to facilitate delivery of at least one substance to a user.

In some embodiments, the delivery system is a non-combustible aerosol provision system, such as a powered non-combustible aerosol provision system.

In some embodiments, the non-combustible aerosol provision system is an electronic cigarette, also known as a vaping device or electronic nicotine delivery system (END), although it is noted that the presence of nicotine in the aerosol-generating material is not a requirement.

In some embodiments, the non-combustible aerosol provision system is an aerosol-generating material heating system, also known as a heat-not-burn system. An example of such a system is a tobacco heating system.

In some embodiments, the non-combustible aerosol provision system is a hybrid system to generate aerosol using a combination of aerosol-generating materials, one or a plurality of which may be heated. Each of the aerosol-generating materials may be, for example, in the form of a solid, liquid or gel and may or may not contain nicotine. In some embodiments, the hybrid system comprises a liquid or gel aerosol-generating material and a solid aerosol-generating material. The solid aerosol-generating material may comprise, for example, tobacco or a non-tobacco product.

Typically, the non-combustible aerosol provision system may comprise a non-combustible aerosol provision device and a consumable for use with the non-combustible aerosol provision device.

In some embodiments, the disclosure relates to consumables comprising aerosol-generating material and configured to be used with non-combustible aerosol provision devices. These consumables are sometimes referred to as articles throughout the disclosure.

In some embodiments, the non-combustible aerosol provision system, such as a non-combustible aerosol provision device thereof, may comprise a power source and a controller. The power source may, for example, be an electric power source or an exothermic power source. In some embodiments, the exothermic power source comprises a carbon substrate which may be energized so as to distribute power in the form of heat to an aerosol-generating material or to a heat transfer material in proximity to the exothermic power source.

In some embodiments, the non-combustible aerosol provision system may comprise an area for receiving the consumable, an aerosol generator, an aerosol generation area, a housing, a mouthpiece, a filter and/or an aerosol-modifying agent.

In some embodiments, the consumable for use with the non-combustible aerosol provision device may comprise aerosol-generating material, an aerosol-generating material storage area, an aerosol-generating material transfer component, an aerosol generator, an aerosol generation area, a housing, a wrapper, a filter, a mouthpiece, and/or an aerosol-modifying agent.

As used herein, the term “aerosol-generating material” includes materials that provide volatilized components upon heating, typically in the form of vapor or an aerosol. “Aerosol-generating material” may be a non-tobacco-containing material or a tobacco-containing material. “Aerosol-generating material” may, for example, include one or more of tobacco per se, tobacco derivatives, expanded tobacco, reconstituted tobacco, tobacco extract, homogenized tobacco or tobacco substitutes. The aerosol-generating material can be in the form of ground tobacco, cut rag tobacco, extruded tobacco, reconstituted tobacco, reconstituted aerosol-generating material, liquid, gel, amorphous solid, gelled sheet, powder, or agglomerates, or the like. “Aerosol-generating material” also may include other, non-tobacco, products, which, depending on the product, may or may not contain nicotine. “Aerosol-generating material” may comprise one or more humectants, such as glycerol or propylene glycol.

As noted above, the aerosol-generating material may comprise an “amorphous solid”, which may alternatively be referred to as a “monolithic solid” (i.e. non-fibrous), or as a “dried gel”. The amorphous solid is a solid material that may retain some fluid, such as liquid, within it. In some cases, the aerosol-generating material comprises from about 50 wt %, 60 wt % or 70 wt % of amorphous solid, to about 90 wt %, 95 wt % or 100 wt % of amorphous solid. In some cases, the aerosol-generating material consists of amorphous solid.

As used herein, the term “sheet” denotes an element having a width and length substantially greater than a thickness thereof. The sheet may be a strip, for example.

As used herein, the term “heating material” or “heater material” refers to material that is heatable by penetration with a varying magnetic field.

A susceptor is a material that is heatable by penetration with a varying magnetic field, such as an alternating magnetic field. The susceptor may be an electrically-conductive material, so that penetration thereof with a varying magnetic field causes induction heating of the heating material. The heating material may be magnetic material, so that penetration thereof with a varying magnetic field causes magnetic hysteresis heating of the heating material. The susceptor may be both electrically-conductive and magnetic, so that the susceptor is heatable by both heating mechanisms. The device that is configured to generate the varying magnetic field is referred to as a magnetic field generator, herein.

It has been found that, when the susceptor is in the form of a closed electrical circuit, magnetic coupling between the susceptor and the electromagnet in use is enhanced, which results in greater or improved Joule heating.

An aerosol generator is an apparatus configured to cause aerosol to be generated from the aerosol-generating material. In some embodiments, the aerosol generator is a heater configured to subject the aerosol-generating material to heat energy, so as to release one or more volatiles from the aerosol-generating material to form an aerosol. In some embodiments, the aerosol generator is configured to cause an aerosol to be generated from the aerosol-generating material without heating. For example, the aerosol generator may be configured to subject the aerosol-generating material to one or more of vibration, increased pressure, or electrostatic energy.

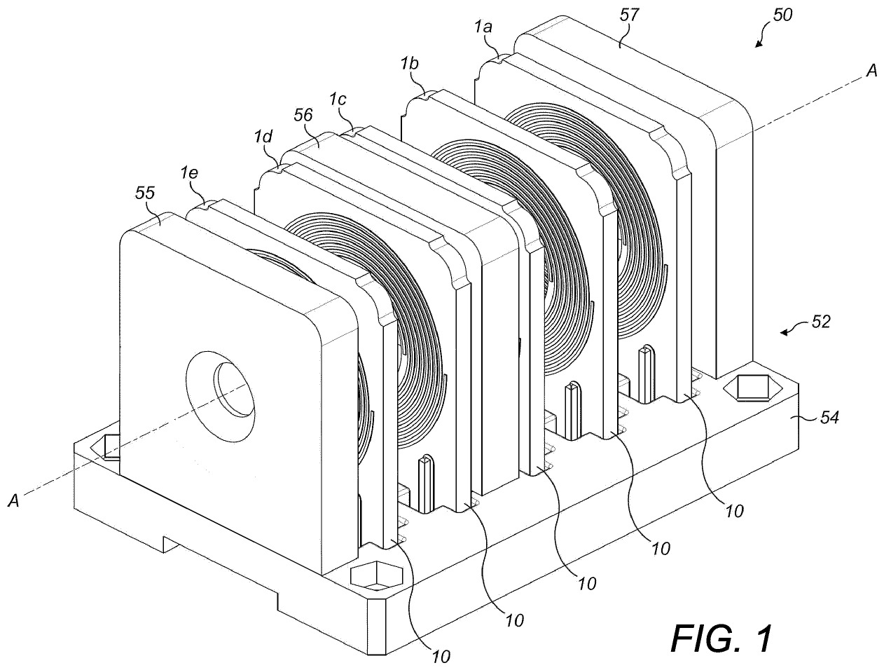

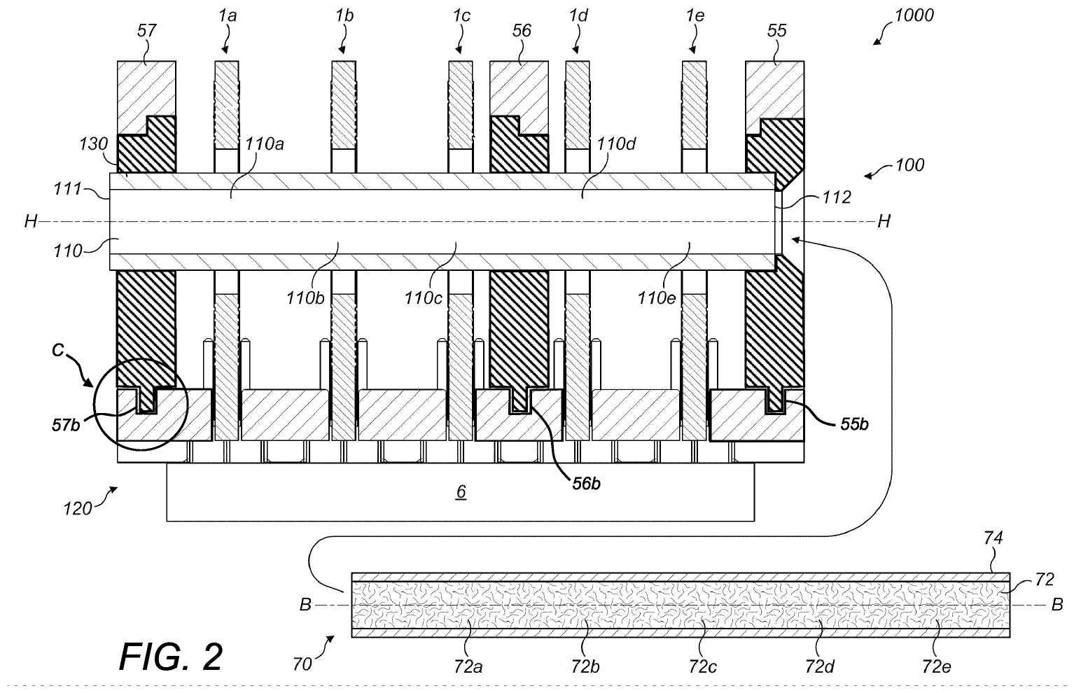

Referring to FIG. 1, there is shown a schematic perspective view of an example of a structure 50 according to an embodiment of the disclosure. Parts of the structure 50 may be comprised in a non-combustible aerosol provision device, such as that 100 shown in FIG. 2 and described below.

The structure 50 of this embodiment comprises first to fifth induction coil arrangements 1a, 1b, 1c, 1d, 1e each comprising a flat spiral induction coil of electrically-conductive material, such as copper, mounted on a side of a board or plate 10. In use, a varying (e.g. alternating) electric current is passed through each of the induction coils so as to create a varying (e.g. alternating) magnetic field that is usable to penetrate a heating element to cause heating of the heating element, as will be described in more detail below. In some embodiments, there may be only one magnetic field generated in the non-combustible aerosol provision device.

The structure 50 comprises a holder 52 to which respective plates 10 of the induction coil arrangements 1a, 1b, 1c, 1d, 1e are attached to fix the induction coil arrangements 1a, 1b, 1c, 1d, 1e in position relative to one another. In this embodiment, each plate 10 is substantially planar. In some embodiments, each plate 10 may be made from a non-electrically-conductive material, such as a plastics material, so as to electrically-insulate the coils of adjacent coil arrangements from each other.

In this embodiment, the holder 52 comprises a base 54 and the induction coil arrangements 1a, 1b, 1c, 1d, 1e extend away from the base 54 in a direction orthogonal or normal to a surface of the base 54.

The holder 52 holds the induction coil arrangements 1a, 1b, 1c, 1d, 1e relative to one another so that the flat spiral coils of the induction coil arrangements 1a, 1b, 1c, 1d, 1e are arranged sequentially and in respective planes along an axis A-A. In this embodiment, the flat spiral coils of the induction coil arrangements 1a, 1b, 1c, 1d, 1e lie in respective substantially parallel planes, each of which is orthogonal to the axis A-A. Further, the flat spiral coils are all axially-aligned with each other, since the respective virtual points from which the paths of the coils emanate all lie on a common axis, in this case the axis A-A.

In this embodiment, the structure 50 comprises a controller (not shown) for controlling operation of the flat spiral coils. The controller is housed in the holder 52 and comprises an integrated circuit (IC), but in other embodiments the controller may take a different form. In some embodiments, the controller is for controlling operation of at least one of the induction coil arrangements 1a, 1b, 1c, 1d, 1e independently of at least one other of the induction coil arrangements 1a, 1b, 1c, 1d, 1e. For example, the controller may supply electrical power to the coils of each of the induction coil arrangements 1a, 1b, 1c, 1d, 1e independently of the coils of the other induction coil arrangements 1a, 1b, 1c, 1d, 1e. In some embodiments, the controller may supply electrical power to the coils of each of the induction coil arrangements 1a, 1b, 1c, 1d, 1e sequentially. Alternatively, in one mode of operation at least, the controller may be for controlling operation of all of the induction coil arrangements 1a, 1b, 1c, 1d, 1e simultaneously.

The holder 52 further comprises three supporting members 55, 56, 57 that extend away from the base 54 when engaged with the base 54 in a direction orthogonal or normal to a surface of the base 54, and substantially parallel to the induction coil arrangements 1a, 1b, 1c, 1d, 1e. In some embodiments, the supporting members 55, 56, 57 may not be comprised by the device 100 but may be combinable with the device 100 to form a non-combustible aerosol provision system 1000 such as the one shown in FIG. 2. However, in the embodiment shown, the structure 50 excluding the supporting members 55, 56, 57 is comprised by the device 100.

The supporting members 55, 56, 57 are insertable by a user. The supporting members 55, 56, 57 can therefore be referred to as user-insertable supports. The supporting members 55, 56, 57 are separate from the device 100 but combinable with the device 100 by engagement. In some embodiments, the engagement involves slidable coupling wherein a supporting member 55, 56, 57 is assembled to the base 54 of the device 100 by a user in a linear direction along the longitudinal axis A-A or in a linear direction radial to the longitudinal axis A-A, for example. In some embodiments, arcuate insertion paths are utilized, and such utilization is determined by the shape of an engagement region of the device 100 and respective engaging portion of the supporting members 55, 56, 57.

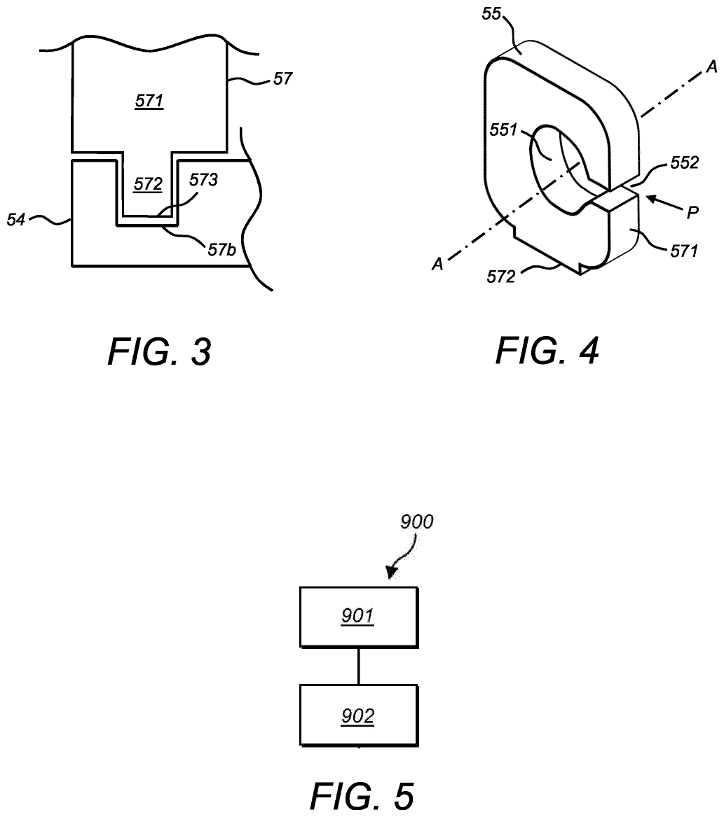

In this embodiment, the supporting members 55, 56, 57 are 3D printed SLS (selective laser sintering) nylon and are couplable to the base 54 of the device 100. The user-insertable supports 55, 56, 57 are shown as separate components from the base 54, which are couplable together with the base 54 by a user of a device comprising the structure 50, hence they are user-insertable. Each of the user-insertable supports 55, 56, 57 are configured for engagement and disengagement with an engagement region of the base 54 (see enlarged schematic cross-sectional side view of area C in FIG. 3) and configured for supporting one or more consumables comprising aerosol-generating material in a heating zone 110. In this embodiment and other embodiments, a heating zone is an aerosol-generation zone. That is, the user-insertable supports 55, 56, 57 are configured to support the weight of a consumable when combined with the device 100, wherein the consumable 70 itself comprises aerosol-generating material. In some embodiments, the consumable comprises a heating element 130. However, in other embodiments, the consumable does not comprise the heating element 130. The engagement region is for establishing coupling of at least one supporting member with the device 100 to allow one or more consumables comprising aerosol-generating material to remain stably positioned in the heating zone 110. The engagement region there facilitates coupling of the at least one supporting member with the device 100. Therefore, any suitable coupling mechanism that keeps the one or more consumables comprising aerosol-generating material to remain stable in the heating zone 110 is suitable. The at least one supporting member can be disengaged with the engagement region when the at least one supporting member is removed for cleaning and/or interchanged with at least one other supporting member.

Each of the user-insertable supports 55, 56, 57 has a passageway therethrough which is provided as an aperture or a hole through each user-insertable support 55, 56, 57. The group of passageways all lie on the same axis A-A as the respective virtual points from which the paths of the coils emanate. The passageway is provided to guide the consumable 70 and/or the heating element 130 in the device 100.

When looking in the direction of axis A-A, the cross-section of each passageway of the user-insertable supports 55, 56, 57 is the same. However, in some embodiments, the cross-section may be different. For example, the cross-section may be circular, triangular, square, rectangular or any regular or irregular polygon. In some embodiments, a passageway of one user-insertable support 55, 56, 57 may be geometrically different to a passageway of another user-insertable support 55, 56, 57. For example, a diameter may be different. Such a variation may be present in a set of user-insertable support 55, 56, 57. For example, a size of each passageway may vary from a first end 111 to a second end 112. The size may increase or decrease from a first end 111 to a second end 112. Additionally, or alternatively, one set of user-insertable supports 55, 56, 57 may have a passageway that is geometrically different to a passageway of another set of user-insertable supports 55, 56, 57. For example, the diameter of a first set of user-insertable supports 55, 56, 57 may be greater than that of a second set of user-insertable supports 55, 56, 57. Each set of user-insertable supports 55, 56, 57 is interchangeably engageable with each corresponding engagement region, wherein only one set of user-insertable supports 55, 56, 57 may be used in the device 100 at a given time. The benefit of providing supporting members that are interchangeable, with different supports having different sized passageways therein is to allow a user wishing to change a consumable size when using the same device. A user can then switch out a supporting member of a first type and insert a supporting member of a second type. In this way, the user can customize what type of consumable the device can receive, and the user can use.

Each set of user-insertable supports 55, 56, 57 as described may be combinable with a heating element 130 that is specifically shaped for each set of user-insertable supports 55, 56, 57. The set of user-insertable supports 55, 56, 57 and specifically configured heating element 130 may form a set of components. A heating element 130 of a first set of components may be geometrically different to a heating element 130 of a second set of components. For example, the difference in geometry may be a thickness or a diameter of the passageways of the user-insertable supports 55, 56, 57.

In some embodiments, the number of user-insertable supports 55, 56, 57 is equal the number of induction coil arrangements 1a, 1b, 1c, 1d, 1e. However, in the embodiment shown, the number of user-insertable supports 55, 56, 57 is lower than the number of induction coil arrangements 1a, 1b, 1c, 1d, 1e. At least one user-insertable support 55, 56, 57 is positioned between an induction coil arrangement 1a, 1b, 1c, 1d, 1e.

Referring to FIG. 2, there is shown a schematic cross-sectional view of an example of a non-combustible aerosol provision system according to an embodiment of the disclosure. The non-combustible aerosol provision system 1000 comprises user-insertable supports 55, 56, 57, and a non-combustible aerosol provision device 100 for generating aerosol from aerosol-generating material 72. Optionally, the non-combustible aerosol provision system 1000 comprises a consumable 70 comprising aerosol-generating material 72, such as that shown in FIG. 2. In this embodiment, the aerosol-generating material 72 comprises tobacco, and the device 100 is a tobacco heating product (also known in the art as a tobacco heating device). The device 100 of this embodiment is an example of a heat-not-burn device.

As shown in FIG. 2, the non-combustible aerosol provision system 1000 comprises a heating element 130 which acts as an elongate support for supporting, in use, the consumable 70 comprising aerosol-generating material. In this embodiment, the heating element 130 is removable from the device 100 and separate to the consumable 70 comprising aerosol-generating material. Additionally, the heating element 130 and consumable 70 may be combinable to form an integral consumable item that is discarded together once used. When the heating element 130 is separate to the consumable 70, the heating element 130 may be fixed to the device 100 and is not readily removable by a user. That is, the heating element 130 may be fixed to the rest of the device 100.

In this embodiment, the heating element 130 is tubular and has a longitudinal axis H-H that is coaxial with the axis A-A. A length of the heating element 130 is therefore greater than a width of the heating element 130 perpendicular to the longitudinal axis A-A. In other embodiments, the heating element 130 is a male member such as a rod, pin or blade and configurable to penetrate the consumable 70 comprising aerosol-generating material when the consumable 70 is received in the heating zone 110. The heating element 130 may be a susceptor that is capable of being inductively heated. However, the heating element 130 is not limited to such a heating method and may be capable of being heated by resistance heating. In some embodiments, the male member is configured to extend along the central axis A-A of the heating zone 110 and automatically penetrate a consumable 70 comprising aerosol-generating material when the consumable 70 is inserted.

In use, the heating element 130 is configurable to extend coaxially through the coils. The heating element 130 can be held in position by the user-insertable supports 55, 56, 57 and extends through the holes in the plurality of flat spiral coils, through the passageways in the user-insertable supports 55, 56, 57, and through the apertures in the plates 10. The user-insertable supports 55, 56, 57 help prevent the heating element 130 contacting the induction coil arrangements 1a, 1b, 1c, 1d, 1e, and particularly the coils thereof. The user-insertable supports 55, 56, 57 provide alignment with the central axis A-A of the induction coil arrangements 1a, 1b, 1c, 1d, 1e. The user-insertable supports 55, 56, 57 are used to locate the heating element 130 in a radial direction. Additionally, the user-insertable supports 55, 56, 57 restrict movement of the heating element 130 by friction for example.

In this embodiment, the heating element 130 comprises heating material that is heatable by penetration with a varying magnetic field to heat an interior volume of the heating element 130. More specifically, in use, the respective varying magnetic fields generated by the coils penetrate the heating element 130. Accordingly, respective portions of the heating element 130 are heatable by penetration with the respective varying magnetic fields. When the consumable 70 is arranged in the interior volume of the heating element 130, the aerosol-generating material in the interior volume is heated. When the heating element 130 is a male member, aerosol-generating material may be arrangeable adjacent an exterior of the heating element 130. In this instance, heat may be passed in an exterior direction away from an external surface of the heating element 130. The heating element 130 may be a collar or shim that is insertable within each passageway of the user-insertable supports 55, 56, 57 and may act as a structural support for aerosol-generating material insertable in a chamber 110. The insertion path may comprise a component that is in a direction parallel to the axis A-A of the induction coil arrangements 1a, 1b, 1c, 1d, 1e. The heating element 130 is therefore an additional support to the consumable 70 that acts as a heatable component in use. The controller 6 may be configured to cause heating of the respective portions of the heating element 130 for example at different respective times, for different respective durations, and/or at different respective rates.

Alternatively, referring to FIG. 4, each user-insertable support 55, 56, 57 may comprise an opening 552 arranged to communicate with the passageway 551. The opening 552 allows the heating element 130 to enter the passageway 551 in an insertion direction P that is transverse to the axis A-A. In this instance, the passageway is suitable for receiving a male member type heating element 130 that is configured to penetrate the aerosol-generating material and be surrounded by the aerosol-generating material rather than enclosing or encasing the aerosol-generating material. The size of the opening 552 therefore may accommodate a heating element 130 but not the combination of a heating element 130 and aerosol-generating material. The opening 552 is generally smaller in size than a size of the passageway 551. When the heating element 130 is a male member as described, the user-insertable support 55 may not be configured to hold the heating element 130 and a part of the device may provide this function instead. The opening 552 may be a cut-out for accessing the passageway in a different direction to the central axis A-A.

In some embodiments, the magnetic field generator 120 comprises an electrical power source (not shown) and a user interface 218 for user-operation of the controller 6. The device 100 of this embodiment further comprises a temperature sensor (not shown) for sensing a temperature of the heating zone 110.

The electrical power source may be a rechargeable battery. In other embodiments, the electrical power source may be other than a rechargeable battery, such as a non-rechargeable battery, a capacitor, a battery-capacitor hybrid, or a connection to a mains electricity supply.

In this embodiment, the aerosol-generating material 72 is in the form of a rod, and the consumable 70 comprises a cover 74 around the aerosol-generating material 72. The cover 74 encircles the aerosol-generating material 72 and helps to protect the aerosol-generating material 72 from damage during transport and use of the consumable 70. The cover 74 may comprise an adhesive (not shown) that adheres the overlapped free ends of the wrapper to each other. The adhesive helps prevent the overlapped free ends of the wrapper from separating. In other embodiments, the adhesive and/or the cover 74 may be omitted. In still other embodiments, the consumable may take a different form to any of those discussed above.

Broadly speaking, the device 100 comprises an elongate chamber or heating zone 110 for receiving the consumable 70, and an aerosol generator configured to cause aerosol to be generated from the consumable 70 comprising aerosol-generating material. In this embodiment, the aerosol generator is a heating device comprising a magnetic field generator 120 for generating varying magnetic fields that penetrate respective portions 110a, 110b, 110c, 110d, 110e of the heating zone 110 in use. In this embodiment, the heating zone 110 comprises a recess for receiving the consumable 70. The consumable 70 may be insertable into the heating zone 110 by a user in any suitable manner, such as through a slot in a wall of the device 100, or by first moving a portion of the device 100, such as a mouthpiece, to access the heating zone 110. In other embodiments, the heating zone 110 may be other than a recess, such as a shelf, a surface, or a projection, and may require mechanical mating with the consumable in order to co-operate with, or receive, the consumable. In this embodiment, the heating zone 110 is sized and shaped to accommodate the whole consumable 70. In other embodiments, the heating zone 110 may be dimensioned to receive only a portion of the consumable 70 in use.

The device 100 has an air inlet (not shown) that fluidly connects the heating zone 110 with the exterior of the device 100, and an outlet (not shown) for permitting volatilized material to pass from the heating zone 110 to an exterior of the device 100 in use. A user may be able to inhale the volatilized component(s) of the aerosol-generating material 72 by drawing the volatilized component(s) through the outlet. As the volatilized component(s) are removed from the heating zone 110, air may be drawn into the heating zone 110 via the air inlet of the device 100. A first end 111 of the heating zone 110 is closest to the outlet, and a second end 112 of the heating zone 110 is closest to the air inlet.

The heating element 130 is open at both the first end 111 and the second end 112 that is opposite the first end 111. The first end 111 therefore comprises a first opening and the second end 112 comprises a second opening. The first and second openings are axially aligned on the axis H-H shown in FIG. 2. The first and second openings are also parallel to one another. Aerosol-generating material is insertable through the first opening to access the chamber 110 of the heating element 130. Therefore, the first opening is the initial point of passage of aerosol-generating material in the chamber 110. The chamber 110 comprises a constant cross-section and extends between the first end 111 and the second end 112 of the heating element 130.

In the orientation shown in FIG. 2, the heating element 130 is generally cylindrical with a substantially circular cross section. In other embodiments, the heating element 130 may have an oval or elliptical cross section or may be other than cylindrical. In some embodiments, the heating element 130 may have a polygonal, quadrilateral, rectangular, square, triangular, star-shaped, or irregular cross section, for example. In this embodiment, the heating element 130 is a tube wherein the body 2 is tubular. The heating element 130 comprises a chamber 110 which is a hollow inner region of the tube. The chamber 110 corresponds to a heating zone when the heating element 130 is arranged in a device 100. The chamber 110 is formed by the body 2 of the heating element 130 and is configured for receiving the aerosol-generating material.

In this embodiment, the consumable 70 is elongate with a longitudinal axis B-B. When the consumable 70 is located in the heating zone 110 in use, this axis B-B lies coaxial with, or parallel to, the longitudinal axis H-H of the heating zone 110. Accordingly, the heating of one or more portion(s) of the heating element 130 causes heating of one or more of the corresponding portion(s) 110a, 110b, 110c, 110d, 110e of the heating zone 110. In turn, this causes heating of one or more corresponding section(s) 72a, 72b, 72c, 72d, 72e of the aerosol-generating material 72 of the consumable 70, when the consumable 70 is located in the heating zone 110. Other portions of the heating element 130 that are adjacent the portion(s) 110a, 110b, 110c, 110d, 110e of the heating zone 110 that are heated in the heating zone 110 are heated by conduction.

Referring to FIG. 3, an enlarged partial schematic cross-sectional side view of the example non-combustible aerosol provision system of FIG. 2 is shown. Although the view of FIG. 3 specifically relates to a first user-insertable support 57 arranged at a first end 111 of the of the heating zone 110, the features of the first user-insertable support 57 may be comprised by at least one other of the user-insertable supports 55, 56.

The first user-insertable support 57 comprises a body 571 and a first coupling member 572. The body 571 comprises the passageway through which aerosol-generating material is insertable. The first coupling member 572 is a radial protrusion from the body 571. That is, when the first user-insertable support 57 is arranged in the base 54, the first coupling member 572 is configured to protrude orthogonally to the longitudinal axis H-H.

The base 54 comprises a second coupling member shown as a recess. However, in other embodiments, the second coupling member is a protrusion that is receivable within the first coupling member 572 when the first coupling member 572 is provided as a recess in the body 571 of the first user-insertable support 57.

An external surface 573 of the first coupling member 572 is abuttable against a corresponding external surface 57a, 57b, 57c of the second coupling member. The second coupling member is configured to prevent movement of the first user-insertable support 57 relative to the base 54 of the holder 52. The second coupling member is therefore an engagement region that is occupiable by at least a part of the first coupling member 572. The engagement region (i.e. the second coupling member) is suitable for restraining movement, e.g. longitudinal movement, of the first user-insertable support 57 relative to the base 54 when the first user-insertable support 57 is installed in the device 100. The engagement region therefore acts as retainer to retain a movement of the first user-insertable support 57 and retain the heating element 130 in the device 100 relative to at least one direction of movement. Such directional movement may be axial movement which is movement in an axial direction of the user-insertable support 55, 56, 57, for example along longitudinal axis H-H, shown in FIG. 2. In some embodiments, the engagement region resists translational movement of the user-insertable support 55, 56, 57 relative to the base 24. The engagement region shown in this embodiment alternatively or additionally resists rotation of the user-insertable support 55, 56, 57 about the longitudinal axis H-H.

The engagement region is an abutment member for abutting at least one surface of a user-insertable support 55, 56, 57 and limiting the extent of movement of the user-insertable support 55, 56, 57. The first coupling member 572 is blockable by a corresponding second coupling member (i.e. engagement region of the device 100) to prevent movement of the user-insertable support 55, 56, 57 in the device 100, particularly when an consumable containing aerosol-generating material is removed from the device 100.

The first coupling member 572 of the user-insertable support 55, 56, 57 is used to hold the user-insertable support 55, 56, 57 in a specific location in the device 100. In some embodiments, the first coupling member 572 engages with the engagement region by a push fit relationship. In this instance, a push fit relationship is when a first member (e.g. the first coupling member or second coupling member) is insertable into a second member (e.g. the other of the first coupling member or second coupling member) using an insertion force. The insertion force is exertable by a user's fingers to overcome frictional resistance between the first and second members. This frictional resistance holds the first and second members together under friction as one combination. Therefore, separation of the first and second members is achieved by exerting a finger force similar to the insertion force. In a push fit relationship, the first and second members are not free to move relative to each other but are also not permanently fixed in position relative to each other because they are moveable relative to one another by finger force. Each of the first coupling member and second coupling remember therefore prevents free movement of the user-insertable support 55, 56, 57 without being fixed in position when a push fit relationship is used. In the embodiment of FIGS. 2 and 3, the second coupling member therefore facilitates improved retention of the user-insertable support 55, 56, 57 in a device.

Alternatively, to a push fit relationship, a screw relationship may be used in other embodiments. For example, the first coupling member 572 may be a threaded rod and the engagement region may be a threaded hole for receiving the threaded rod. Another means of engagement alternatively or additionally to the push fit relationship may be the use of a locking pin insertable into in either or both of the supporting member 55, 56, 57 and engagement region for preventing removal of the supporting member 55, 56, 57 from the engagement region. In some embodiments, the device comprises a plurality of receiving portions each for receiving a locking pin. In such embodiments, the device comprises a single engagement region for engagement with a plurality of sequentially insertable supporting members 55, 56, 57. The locking pin therefore locates the supporting members 55, 56, 57 in use.

In some embodiments, the first coupling members 572 and engagement region interact using a dovetail interlocking relationship. For example, the first coupling member 572 may comprise a dovetail shape which is receivable in a correspondingly shaped engagement region to form a dovetail joint when combined.

In some embodiments, each of the first coupling members 572 has the same general shape. Each of the corresponding second coupling members may have the same general shape. Each of the first coupling members 572 protrudes away from the body 571 of the user-insertable support 55, 56, 57 to a similar extent. In some embodiments, each of the first coupling members 572 extends along a width of the user-insertable support 55, 56, 57 to a similar extent. Each of the second coupling members may extend along a width of the device 100 to a similar extent. However, the length and width of each of the first and second coupling member may vary. At least one corner and/or edge of each coupling member may be chamfered, rounded or beveled.

FIG. 5 shows a flow diagram showing an example of a method 900 of configuring a non-combustible aerosol provision device for generating aerosol from aerosol-generating material. The method comprises providing 901 a non-combustible aerosol provision device for generating the aerosol from aerosol-generating material. The method also comprises user engagement 902 of an engagement region of the device with at least one user-insertable support for supporting one or more consumables comprising aerosol-generating material in a heating zone of the device.

In some embodiments, the user engagement 902 comprises inserting a first coupling member of the at least one user-insertable support into the engagement region of the device. Alternatively, the user engagement 902 may comprise inserting the engagement region of the device into the first coupling member of the at least one user-insertable support. In some embodiments, the user engagement 902 comprises engaging the engagement region of the device with the at least one user-insertable support by push fit such that frictional resistance maintains the user engagement. The user engagement 902 may comprise sequentially inserting a first coupling member of the at least one user-insertable support into a first engagement region of the device and a second coupling member of at least another one user-insertable support into a second engagement region of the device.

The method may further comprise inserting a heating element into an opening of the at least one user-insertable support, wherein the opening is communicable with a passageway of the user-insertable support through which the heating element is insertable along an insertion path. An axis of the opening may be orthogonal to an axis of the passageway. The heating element may be a male member such as a rod, pin or blade and may be configurable to penetrate the one or more consumables comprising aerosol-generating material when the one or more consumables are received by the heating zone. The male member may be configured to extend along the central axis of the heating zone and automatically penetrate a consumable comprising aerosol-generating material when the consumable is inserted.

In some embodiments, the heating material is aluminum. However, in other embodiments, the heating material may be other than aluminum. In some embodiments, the heating material may comprise one or more materials selected from the group consisting of: an electrically-conductive material, a magnetic material, and a magnetic electrically-conductive material. In some embodiments, the heating material may comprise a metal or a metal alloy. In some embodiments, the heating material may comprise one or more materials selected from the group consisting of: aluminum, gold, iron, nickel, cobalt, conductive carbon, graphite, steel, plain-carbon steel, mild steel, stainless steel, ferritic stainless steel, molybdenum, silicon carbide, copper, and bronze. Other heating material(s) may be used in other embodiments.

In some embodiments, the sheet comprising heating material is a free from holes or discontinuities. In some embodiments, the sheet comprising heating material comprises a foil, such as a metal or metal alloy foil, e.g. aluminum foil. However, in some embodiments, the sheet comprising heating material may have holes or discontinuities. For example, in some embodiments, the sheet comprising heating material may comprise a mesh, a perforated sheet, or a perforated foil, such as a metal or metal alloy perforated foil, e.g. perforated aluminum foil.

In some embodiments, such as those in which the heating material comprises iron, such as steel (e.g. mild steel or stainless steel) or aluminum, the sheet comprising heating material may be coated to help avoid corrosion or oxidation of the heating material in use. Such coating may, for example, comprise nickel plating, gold plating, or a coating of a ceramic or an inert polymer. In some embodiments, the sheet comprising heating material comprises or consists of nickel-plated aluminum foil.

The heating material may have a skin depth, which is an exterior zone within which most of an induced electrical current and/or induced reorientation of magnetic dipoles occurs. By providing that the heating material has a relatively small thickness, a greater proportion of the heating material may be heatable by a given varying magnetic field, as compared to heating material having a depth or thickness that is relatively large as compared to the other dimensions of the heating material. Thus, a more efficient use of material is achieved and, in turn, costs are reduced.

In some embodiments, the aerosol-generating material comprises tobacco. However, in other embodiments, the aerosol-generating material may consist of tobacco, may consist substantially entirely of tobacco, may comprise tobacco and aerosol-generating material other than tobacco, may comprise aerosol-generating material other than tobacco, or may be free from tobacco. In some embodiments, the aerosol-generating material may comprise a vapor or aerosol forming agent or a humectant, such as glycerol, propylene glycol, triacetin, or diethylene glycol.

In some embodiments, the aerosol-generating material is non-liquid aerosol-generating material, and the device is for generating aerosol from non-liquid aerosol-generating material. Once all, or substantially all, of the volatilizable component(s) of the aerosol-generating material in the consumable 70 has/have been spent, the user may remove the consumable 70 from the heating zone 110 of the device 100 and dispose of the consumable 70. The user may subsequently re-use the device 100 with another of the consumables 70. However, in other respective embodiments, the device 100 and the consumable 70 may be disposed of together once the volatilizable component(s) of the aerosol-generating material has/have been spent.

In some embodiments, the consumable 70 is sold, supplied or otherwise provided separately from the device 100 with which the consumable 70 is usable. However, in some embodiments, the device 100 and one or more of the consumables 70 may be provided together as a non-combustible aerosol provision system, such as a kit or an assembly, possibly with additional components, such as cleaning utensils.

In order to address various issues and advance the art, the entirety of this disclosure shows by way of illustration and example various embodiments in which that which is claimed may be practiced and which provide for superior non-combustible aerosol provision devices for generating aerosol from aerosol-generating material, methods of configuring non-combustible aerosol provision devices for generating aerosol from aerosol-generating material, and non-combustible aerosol provision systems for generating aerosol from aerosol-generating material. The advantages and features of the disclosure are of a representative sample of embodiments only, and are not exhaustive and/or exclusive. They are presented only to assist in understanding and teach the claimed and otherwise disclosed features. It is to be understood that advantages, embodiments, examples, functions, features, structures and/or other aspects of the disclosure are not to be considered limitations on the disclosure as defined by the claims or limitations on equivalents to the claims, and that other embodiments may be utilized and modifications may be made without departing from the scope and/or spirit of the disclosure. Various embodiments may suitably comprise, consist of, or consist in essence of, various combinations of the disclosed elements, components, features, parts, steps, means, etc. The disclosure may include other inventions not presently claimed, but which may be claimed in future.

1. A non-combustible aerosol provision device for generating aerosol from aerosol-generating material, the non-combustible aerosol provision device comprising:
an aerosol-generation zone for receiving one or more consumables comprising aerosol-generating material;
an aerosol generator configured to cause aerosol to be generated from the one or more consumables comprising the aerosol-generating material in the aerosol-generation zone; and
an engagement region for engagement and disengagement with at least one user-insertable support for supporting the one or more consumables comprising the aerosol-generating material in the aerosol-generation zone.

2. The non-combustible aerosol provision device of claim 1, wherein the aerosol generator comprises, or is, a heating device for use in heating the one or more consumables comprising the aerosol-generating material in the aerosol-generation zone.

3. The non-combustible aerosol provision device of claim 2, wherein the heating device comprises a magnetic field generator for generating one or more varying magnetic fields that penetrate one or more respective longitudinal portions of the aerosol-generation zone in use.

4. The non-combustible aerosol provision device of claim 3, wherein:
the magnetic field generator comprises a plurality of flat spiral coils of electrically-conductive material arranged sequentially and in respective planes along a longitudinal axis of the aerosol-generation zone; or
the magnetic field generator comprises a plurality of flat spiral coils of electrically-conductive material arranged sequentially and in respective planes along a longitudinal axis of the aerosol-generation zone and the aerosol-generation zone extends through a hole in each of the plurality of flat spiral coils.

5. (canceled)

6. The non-combustible aerosol provision device of claim 3, wherein:
the heating device comprises a heating element comprising heating material that is heatable by penetration with a varying magnetic field generatable by the magnetic field generator to heat the aerosol-generation zone, or
the heating device comprises a heating element comprising heating material that is heatable by penetration with a varying magnetic field generatable by the magnetic field generator to heat the aerosol-generation zone, and the heating element is tubular and configured to be around the one or more consumables comprising the aerosol-generating material when the one or more consumables are received by the aerosol-generation zone; or
the heating device comprises a heating element comprising heating material that is heatable by penetration with a varying magnetic field generatable by the magnetic field generator to heat the aerosol-generation zone, and the heating element is a male member and configured to penetrate the one or more consumables comprising the aerosol-generating material when the one or more consumables are received by the aerosol-generation zone.

7. (canceled)

8. (canceled)

9. A non-combustible aerosol provision system for generating aerosol from aerosol-generating material, the non-combustible aerosol provision system comprising:
at least one user-insertable support; and
a non-combustible aerosol provision device comprising:
an aerosol generation zone for receiving one or more consumables comprising aerosol-generating material,
an aerosol generator configured to cause aerosol to be generated from the one or more consumables comprising the aerosol-generating material in the aerosol-generation zone, and
an engagement region;
wherein the at least one user-insertable support is engageable and disengageable with the engagement region and is for supporting the one or more consumables comprising the aerosol-generating material in the aerosol-generation zone.

10. The non-combustible aerosol provision system of claim 9, wherein the at least one user-insertable support comprises a passageway to form at least a part of the aerosol-generation zone for receiving the one or more consumables comprising the aerosol-generating material.

11. The non-combustible aerosol provision system of claim 9, wherein the aerosol generator comprises, or is m a heating device for use in heating the one or more consumables comprising the aerosol-generating material in the aerosol-generation zone.

12. The non-combustible aerosol provision system of claim 11, wherein the heating device comprises a magnetic field generator for generating one or more varying magnetic fields that penetrate one or more respective longitudinal portions of the aerosol-generation zone in use.

13. The non-combustible aerosol provision system of claim 12, wherein:
the magnetic field generator comprises a plurality of flat spiral coils of electrically-conductive material arranged sequentially and in respective planes along a longitudinal axis of the aerosol-generation zone; or
the magnetic field generator comprises a plurality of flat spiral coils of electrically-conductive material arranged sequentially and in respective planes along a longitudinal axis of the aerosol-generation zone and the aerosol-generation zone extends through a hole in each of the plurality of flat spiral coils.

14. (canceled)

15. The non-combustible aerosol provision system of claim 11, wherein the non-combustible aerosol provision system comprises a heating element comprising heating material that is heatable by penetration with a varying magnetic field generatable by a magnetic field generator to heat the aerosol-generation zone.

16. The non-combustible aerosol provision system of claim 15, wherein the non-combustible aerosol provision device comprises the heating element.

17. The non-combustible aerosol provision system of claim 16, wherein:
the heating element is a male member and configured to penetrate the one or more consumables comprising aerosol-generating material when the one or more consumables are received by the aerosol-generation zone; or
the heating element is a male member and configured to penetrate the one or more consumables comprising aerosol-generating material when the one or more consumables are received by the aerosol-generation zone and the at least one user-insertable support comprises an opening communicable with a passageway through which the heating element as a male member is insertable along an insertion path.

18. (canceled)

19. The non-combustible aerosol provision system of claim 9, wherein the non-combustible aerosol provision system comprises a first user-insertable support and a second user-insertable support, wherein the first user-insertable support and the second user-insertable support are interchangeably insertable in the non-combustible aerosol provision device by a user of the non-combustible aerosol provision system.

20. The non-combustible aerosol provision system of claim 19, wherein the first user-insertable support comprises a first passageway to form at least a part of a first aerosol-generation zone and the second user-insertable support comprises a second passageway to form at least a part of a second aerosol-generation zone and wherein the first passageway is geometrically different from the second passageway.

21. The non-combustible aerosol provision system of claim 20, wherein the first user-insertable support and a first heating element form a first set of components and the second user-insertable support and a second heating element form a second set of components, wherein the first heating element and the second heating element each comprise heating material that is heatable by penetration with a varying magnetic field generatable by a magnetic field generator to heat the respective first aerosol-generation zone and the second aerosol-generation zone.

22. A method of configuring a non-combustible aerosol provision device for generating aerosol from aerosol-generating material, the method comprising:
providing a non-combustible aerosol provision device for generating aerosol from aerosol-generating material; and
user engagement of an engagement region of the non-combustible aerosol provision device with at least one user-insertable support for supporting one or more consumables comprising the aerosol-generating material in an aerosol-generation zone of the non-combustible aerosol provision device.

23. A non-combustible aerosol provision system for generating aerosol from aerosol-generating material, the non-combustible aerosol provision system comprising:
an aerosol generator for use in generating aerosol from one or more consumables comprising aerosol-generating material; and
a plurality of adapters interchangeably couplable to the aerosol generator by a user of the non-combustible aerosol provision system;
wherein each adapter of the plurality of adapters is differently dimensioned to support a respectively differently dimensioned consumable comprising the aerosol-generating material in the aerosol generator.

24. The non-combustible aerosol provision system of claim 23, wherein the aerosol generator comprises a plurality of spiral coils of electrically-conductive material spaced apart from each other along an axis of the aerosol generator for generating respective varying magnetic fields.

25. The non-combustible aerosol provision system of claim 23, wherein the aerosol generator is a heater for heating the aerosol-generating material in thermal contact with the heater.
